(19) United States
(12) Patent Application Publication
(10) Pub. No.: US 2004/0073007 A1
(43) Pub. Date: Apr. 15, 2004

(54) Antiangiogenic peptides

(76) Inventors: Francesco Chillemi, Milano (IT); Lucia Maria Teresa Vicentini, Milano (IT); Pierangelo Francescato, Milano (IT)

Correspondence Address: YOUNG & THOMPSON, 745 SOUTH 23RD STREET 2ND FLOOR, ARLINGTON, VA, 22202

(21) Appl. No.: 10/468,759

(22) PCT Filed: Feb. 27, 2002
(86) PCT No.: PCT/IT02/00119

(30) Foreign Application Priority Data
Feb. 27, 2001 (IT) MI2001A000394

(51) Int. Cl. C07K 1/00; C07K 14/00; C07K 17/00

(52) U.S. Cl. 530/350

(57) ABSTRACT

Peptides with antiangiogenic activity with a sequence corresponding to that of fragments of human endostatin.

The present invention relates to peptides, With antiangiogenic activity having a sequence corresponding to fragments of human endostatin.

### BACKGROUND OF THE INVENTION

Angiogenesis is the process of outgrowth of new capillaries from pre-existing blood vessels. This phenomenon occurs in various physiological and pathological conditions and is particularly involved in tumor growth and in formation and maintenance of metastasis.

Angiogenesis is a complex multistep process that includes proliferation, migration and differentiation of endothelial cells, with parallel degradation events of the extra-cellular matrix, formation of tubules and “sprouting” of new capillaries.

Endostatin is a C-terminal fragment of XCIII collagen with molecular weight of 20 kDa, that specifically inhibits endothelial cells proliferation in vitro and angiogenesis and tumor growth in vivo. In particular, systemic administration of recombinant endostatin causes regression of tumors in mice. However, administration—and consequently production—of large amounts of endostatin are necessary to observe these effects. Moreover, the protein is unstable and, when recombinantly produced in E. Coli, solubility problems arise. Availability of molecules endowed with biological activity comparable to endostatin, but having smaller dimensions and higher stability and solubility, may be extremely useful.

Peptides with a sequence corresponding to murine endostatin described by Folkman in WO 97/15666, have been disclosed in WO 99/29855, WO 99/48924 and WO 00/63249.

In particular, WO 99/29855 (Beth Israel Deaconess Medical Center) discloses mutants and peptides of murine endostatin (deletion of 9 amino acids 176-184 in the C-terminal region) and characterized by the sequence SYIVLCIE (168-175) in the C-terminal region.

WO 99/48924 (Children's Medical Center, Ben-Sasson) discloses peptides having from about 10 to about 28 amino acids deriving from the AHR sequence (angiogenic homology region), corresponding to the 36-70 region of human endostatin. Hybrid peptides containing 10-11 amino acids corresponding to the endostatin AHR sequence and other 10-11 amino acids corresponding to the AHR sequence of other proteins (TSP-1, TSP-4; TSP=thrombospondin) are therein described in detail.

Finally, WO 00/63249, in the Applicant's name, discloses the fragments corresponding to the sequences 1-39, 40-89, 90-134, 135-184 of murine endostatin. Some of said fragments are more active than the whole endostatin molecule.

The murine sequence has about 86% homology with the human sequence.

### DISCLOSURE OF THE INVENTION

It has now been found that some peptides having from 20 to 50 amino acids with sequences corresponding to the sequence 6-179 of human endostatin show antiangiogenic activity markedly higher than endostatin itself and than the above cited known peptides.

Therefore the invention relates to said peptides, pharmaceutical compositions containing them and the use thereof for the preparation of medicaments with antiangiogenic activity.

### DESCRIPTION OF THE FIGURES

FIG. 1 shows the human endostatin sequences in comparison with the murine;

FIG. 2 shows the percentage inhibition curves of cellular migration obtained with peptide 6-49 in comparison with human endostatin;

FIGS. 3a and 3b show the graphic representation of the percent inhibition of DNA synthesis in endothelial human cells Eahy 926 by peptide 6-49 and by human endostatin respectively;

FIG. 4 shows the percent inhibition of tubules formation by peptide 6-49 in the in vivo Matrigel assay;

FIG. 5 shows the results obtained in the in vitro Matrigel assay, by measuring the hemoglobin amount in the gelatinized pellet implanted in C57/b16 mice treated with peptide 6-49.

### DETAILED DISCLOSURE OF THE INVENTION

The peptides of the invention have a sequence from 20 to 50. preferably from 30 to 45, neighboring amino acids of any region of the sequence 6-179 of human endostatin. The invention also comprises the derivatives of said peptides obtained by substitution of natural amino acids with the corresponding amino acids of the D series and/or by derivatization of hydroxy, thio or amino functional groups of serine, threonine, cysteine, tyrosine, lysine, arginine residues and/or by functionalization of the terminal NH₂ (for example, by acylation with acetyl groups) and/or by retro-inversion of one or more peptide bonds, according to known techniques which allow to stabilize peptides against hydrolytic enzymes, therefore improving the pharmacokinetic characteristics.

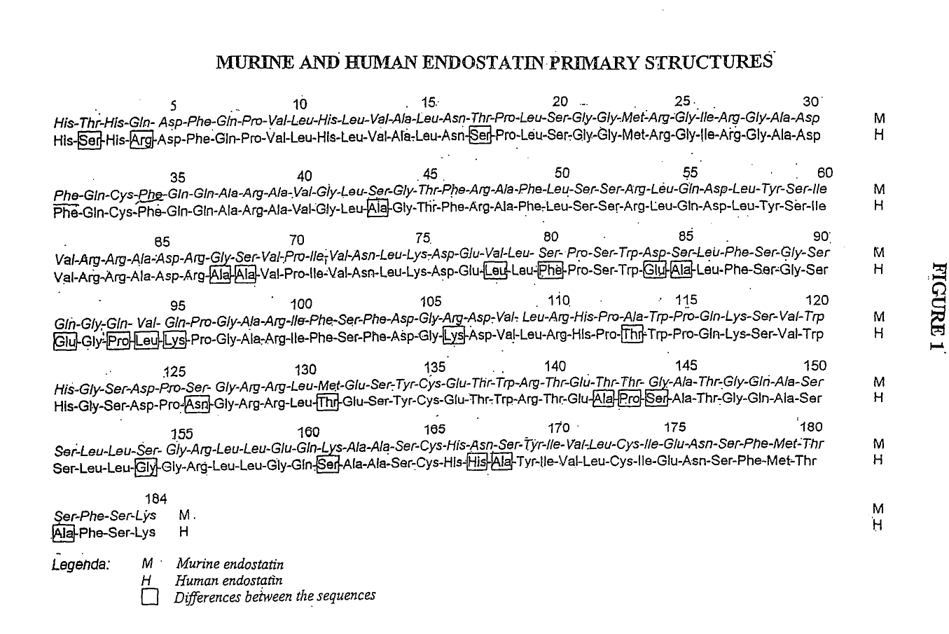

Examples of peptides of the invention are, with reference to the human endostatin sequence reported in FIG. 1, those defined by the sequences 6-40. 6-49, 7-42, 8-52, 10-44, 10-47, 11-64, 12-43,13-50. 15-55, 17-47, 25-65, 33-77, 41-80. 49-88, 50-92, 70-117, 88-110. 90-127, 93-133, 111-150. 124-161, 130-170. 133-179, etc.

Particularly preferred peptides are those defined by the sequence ranging from the amino acids 6-49, 11-64, 50-92, 93-133 and 134-179 of the human endostatin sequence.

Peptides with sequence ranging from the amino acids 6 to 92 of the human sequence are particularly preferred, more preferably those with sequence ranging from the amino acids 6 to 64.

Peptide 6-49 is most preferred.

The peptides object of the present invention can be prepared with methods and reactions conventionally used in the peptide synthesis.

The protection of the amino groups in the amino acids can be carried out by use of 9-fluorenylmethoxycarbonyl (Fmoc), tert-butoxycarbonyl (Boc), benzyloxycarbonyl (Z), trityl (Trt) moieties and others commonly used in the peptide chemistry.

The carboxylic group can be protected by means of the tert-butyl ester, benzyl ester, p-methoxybenzyl ester and others conventionally used for said purposes.

These protective groups can be removed according to processes known in literature, such as by treatment with trifluoroacetic acid, anhydrous hydrofluoric acid, piperidine and the like.

The amino acids can be condensed by using active esters such as pentafluorophenyl ester (OPfp), 3-hydroxy-4-oxo-3,4-dihydro-1,2,3-benzotriazine ester (ODhbT), or carboxy-activators such as benzotriazol-1-yl-oxy-tris-pyrrolidino-phosphonium hexafluorophosphate (PyBop), 2-(1H-benzotriazol-1-yl-1,1,3,3-tetramethyl)-uroniumn tetrafluoroborate (TBTU) and the other activators conventionally used for this type of reactions.

The purification of the polypeptides described in the present invention can also be carried out according to known techniques of protein chemistry, such as reverse phase HPLC, gel filtration, ion exchange chromatography and preparative electrophoresis.

More particularly, the peptides can be prepared using the solid phase peptide synthesis and the automatic synthesizer Biolynx plus, mod. 4170 by Novabiochem (Nottingham, Great Britain) (A. Dryland and R. C. Sheppard, J. Chem. Soc., Perkin 1, 125, 1986).

The protection of the α-amino groups in the amino acids can be carried out by use of 9-fluorenylmethoxycarbonyl (Fmoc). The functional groups of the amino acids side chains are protected using the following protective groups: tert-butyl for aspartic acid, glutamic acid, serine, threonine and tyrosine; tert-butoxycarbonyl for lysine and trypthophan; trityl for histidine; 2,2,4,6,7-pentamethyl-dihydro-benzofuran-5-sulfonyl for arginine; tert-butyl and trityl for cysteine.

The synthesis is gradually carried out starting from the C-terminal Fmoc-amino acid, attached by an ester bond to a resin consisting of polyethylene oxide grafted to a polystyrene matrix and functionalized by a 4-hydroxymethyl-phenoxyacetic acid residue (E. Bayer, Angew. Chem., 103, 117, 1991). Fmoc is removed by using a solution of piperidine in dimethylformamide (DMF). Pentafluorophenyl esters of Fmoc-amino acids are generally used for the condensation reactions. In the case of serine and threonine, the use of ODhbt esters was preferred, whereas in the case of arginine and histidine the carboxylic group was activated by PyBop in the presence of diisopropylethylamine, with three hour reaction times. To maximize the reaction yields, a five equivalent excess of Fmoc-amino acid is used. The times of deprotection and condensation reactions are automatically determined by the synthesizer; the technician will select the acylation times only in the case of activation with PyBop.

The peptide is cleaved from the solid carrier, at the same time removing all the protective groups, by acidolysis with a mixture having the following composition: 80% TFA, 5% H₂O, 2.5% ethanedithiol, 2.5% phenol and 5% thioanisole.

The resulting crude polypeptides are purified by reverse phase semipreparative HPLC, using a column “Jupiter” (250×10 mm) C₁₈, 10μ (Phenomenex, U.S.A.) and an Aktabasic apparatus 100 mod. 18-1405 (Amersham Pharmacia Biotech, Freiburg). Solvent A: 90% of 0.1% trifluoroacetic acid and 10% of acetonitrile; solvent B: 90% of acetonitrile and 10% of 0.1% trifluoroacetic acid. Gradient: from solvent A to solvent B in 65 minutes. Flow rate: 5 ml/minute. Detection at λ=226 nm. 20-25 mg of product are loaded for each run.

The main fractions are collected and freeze-dried.

The purified polypeptides are characterized by amino acid analysis and electrospray mass spectrometry with a Finnigan Mat apparatus mod. LCQ.

The peptides of the invention were found to be particularly active as angiogenesis inhibitors, as evidenced in cell migration, chemotaxis and proliferation tests using EAhy 926 human endothelial cells as well as in assays based on the use of Matrigel in vitro and in vivo.

For the envisaged therapeutical uses, the peptides of the invention or the salts or non toxic derivatives thereof will be suitably formulated in pharmaceutical compositions in admixture with a suitable diluent or carrier. The peptide compositions will be usually administered parenterally, albeit other administration routes, such as the oral, rectal, sublingual or transdermal, are not excluded. Dosages will depend on a number of factors and they will be easily determined by those skilled in the art, according to the case. Anyway a dosage range from about 0.01 to about 1 mg/kg/day may be contemplated.

The following examples will illustrate the invention in greater detail.

### EXAMPLE 1

Phe-Gln-Pro-Val-Leu-His(Trt)-Leu-Val-Ala-Leu-Asn-Ser(tBu)-Pro-Leu-Ser(tBu)-Gly-Gly-Met-Arg(Pbf)-Gly-Ile-Arg(Pbf)-Gly-Ala-Asp(OtBu)-Phe-Gln-Cys(Acm)-Phe-Gln-Gln-Ala-Arg(Pbf)-Ala-Val-Gly-Leu-Ala-Gly-Thr(tBu)-Phe-Arg(Pbf)-Ala-Phe-resin.

666 mg (0.1 ml) of Fmoc-Phe-resin were suspended in 25 ml of DMF and after 2 hours they were loaded into the reaction column.

The Fmoc-amino acid-resin was then subjected to the following treatments: a) washings with DMF; b) removal of Fmoc by treatment with a 20% piperidine solution in DMF; c) washings with DMF; d) condensation with the suitable. Fmoc-amino acid active ester (5 equivalents) in the presence of N-hydroxy-benzotriazole (5 equivalents) as catalyst, with the addition of an anionic dye (Novachrome, Calbiochem-Novabiochem AG, Laufelfingen, Switzerland) for automatically monitoring the reaction time.

The carboxyl was activated by using PyBop, without addition of dye, only in the case of Fmoc-Arg(Pbf) and Fmoc-His(Trt).

This cycle of operations was repeated with the suitable Fmoc-amino acid to finally obtain the protected resin-tetratetracontapeptide. The product was then placed in a sintered glass funnel and washed in succession with DMF, tert-amyl alcohol, acetic acid, tert-amyl alcohol, methylene chloride, and ethyl ether.

1211 mg of the protected resin-tetratetracontapeptide were obtained.

### EXAMPLE 2

Phe-Gln-Pro-Val-Leu-His-Leu-Val-Ala-Leu-Asn-Ser-Pro-Leu-Ser-Gly-Gly-Met-Arg-Gly-Ile-Arg-Gly-Ala-Asp-Phe-Gln-Cys(Acm)-Phe-Gln-Gln-Ala-Arg-Ala-Val-Gly-Leu-Ala-Gly-Thr-Phe-Arg-Ala-Phe.

1211 mg of protected resin-tetratetracontapeptide were suspended in 200 ml of a mixture having the following composition: 80% TFA, 5% H₂O, 2.5% ethanedithiol, 2.5% phenol and 5% thioanisole; the mixture was reacted for 2 hours with occasional stirring. After filtration under vacuum, the resin was washed with TFA (2×50 ml). The filtrate was slowly added with dry ethyl ether to precipitate the polypeptide. The product was filtered, repeatedly washed with dry ethyl ether and finally dried under vacuum over KOH.

The crude compound was purified by semipreparative HPLC as described above, to obtain 97 mg of pure tetratetracontapeptide.

[α]²⁰_{D}−74.5° (c=0.1 water).

Mass spectrum: molecular peak (M+1)=4778 Da.

Amino acid analysis: Asp=2.10 (2); Thr=0.98 (1); Ser=1.99 (2); Glu=4.11 (4); Pro=1.82 (2); Gly=6.21 (6); Ala=5.96 (6); Cys=0.96 (1); Val=2.98 (3); Met=0.95 (1); Ile=1.1 (1); Leu=4.93 (5); Phe=4.8 (5); His=0.89 (1); Arg=3.96 (4).

### EXAMPLE 3

Leu-Ser(tBu)-Ser(tBu)-Arg(Pbf)-Leu-Gln-Asp(OtBu)-Leu-Tyr(tBu)-Ser(tBu)-Ile-Val-Arg(Pbf)-Arg(Pbf)-Ala-Asp(OtBu)-Aig(Pbf)-Ala-Ala-Val-Pro-Ile-Val-Asn-Leu-Lys(Boc)-Asp(OtBu)-Glu(OtBu)-Leu-Leu-Phe-Pro-Ser-(tBu)-Trp(Boc)-Glu(OtBu)-Ala-Leu-Phe-Ser(tBu)-Gly-Ser(tBu)-Glu(OtBu)-Gly-resin.

454 mg (0.1 mmoles) of Fmoc-Gly-resin were suspended in 25 ml of DMF and after two hours placed in the reaction column.

The operations described in-example 1 were then repeated using the suitable Fmoc-amino acid in each run.

In this synthesis also, the Fmoc-Arg(Pbf) and Fmoc-His(Trt) carboxylic groups were activated with PyBop, those of Fmoc-Ser(tBu) and Fmoc-Thr(tBu) with Dhbt ester, whereas for all the other Fmoc-amino acids the Pfp ester was used. After assembling all of the amino acids, the product was washed as described in example 1, and dried under vacuum.

1190 mg of protected resin-tritetracontapeptide were obtained.

### EXAMPLE 4

Leu-Ser-Ser-Arg-Leu-Gln-Asp-Leu-Tyr-Ser-Ile-Val-Arg-Arg-Ala-Asp-Arg-Ala-Ala-Val-Pro-Ile-Val-Asn-Leu-Lys-Asp-Glu-Leu-Leu-Phe-Pro-Ser-Trp-Glu-Ala-Leu-Phe-Ser-Gly-Ser-Glu-Gly.

1190 mg of protected resin-tritetracontapeptide were treated with 200 ml of a mixture of the following composition: 80% TFA; 5% H₂O; 2.5% ethanedithiol; 2.5% phenol and 5% thioanisole and the procedure described in example 2 was followed.

The crude polypeptide was purified by semipreparative HPLC as described above, to obtain 81 mg of pure tritetracontapeptide.

[α]²⁰_{D}−71.2° (c=0.1 water).

mass spectrum: molecular peak (M+1)=4821 Da.

analysis of the amino acids: Asp=3.89 (4); Ser=5.82 (6); Glu=4.05 (4); Pro=1.96 (2); Gly=2.09 (2); Ala=3.95 (4); Val=3.03 (3); Ile=1.96 (2); Leu 6.87 (7); Tyr=0.98 (1); Phe=2.07 (2); Lys=1.05 (1); Arg=3.87 (4); Trp=1.1 (1).

### EXAMPLE 5

Pro-Leu-Lys(Boc)-Pro-Gly-Ala-Arg(Pbf)-Ile-Phe-Ser(tBu)-Phe-Asp(OtBu)-Gly-Lys(Boc)-Asp(OtBu)-Val-Leu-Arg(Pbf)-His(Trt)-Pro-Thr(tBu)-Trp(Boc)-Pro-Gln-Lys(Boc)-Ser(tBu)-Val-Trp(Boc)-His(Trt)-Gly-Ser(tBu)-Asp(OtBu)-Pro-Asn-Gly-Arg(Pbf)-Arg(Pbf)-Leu-Thr(tBu)-Glu(OtBu)-Ser(tBu)-resin.

526 mg (0.1 mmoles) of Fmoc-Ser(tBu)-resin were suspended in 25 ml of DMF and after 2 hours they were loaded into the reaction column. Then the cycle of operations described in example 1 was repeated, using the suitable Fmoc-amino acid in each run, in the order indicated in the sequence reported above.

1081 mg of protected resin-monotetracontapeptide were obtained.

### EXAMPLE 6

Pro-Leu-Lys-Pro-Gly-Ala-Arg-Ile-Phe-Ser-Phe-Asp-Gly-Lys-Asp-Val-Leu-Arg-His-Pro-Thr-Trp-Pro-Gln-Lys-Ser-Val-Trp-His-Gly-Ser-Asp-Pro-Asn-Gly-Arg-Arg-Leu-Thr-Glu-Ser.

1081 mg of protected resin-monotetracontapeptide were suspended in 200 ml of a mixture having the following composition: 80% TFA, 5% H₂O, 2.5% ethanedithiol, 2.5% phenol and 5% thioanisole. The mixture was reacted for three hours with occasional stirring.

The crude product was purified by semipreparative HPLC as described above, to obtain 101 mg of pure monotetracontapeptide.

[α]²⁰_{D}−77.0° (c=0.1 water).

Mass spectrum: molecular peak (M+1)=4672 Da.

Analysis of the amino acids: Asp=3.88 (4); Thr=2.03 (2); Ser=3.95 (4); Glu=2.01 (2); Pro=4.87 (5); Gly=4.12 (4); Ala=0.99 (1); Val=2.10 (2); Ile=1.02 (1); Leu=3.07 (3); Phe=1.97 (2); Lys=3.07 (3); Arg=3.88 (4); Trp=2.12 (2).

### EXAMPLE 7

Tyr(tBu)-Cys(Trt)-Glu(OtBu)-Thr(tBu)-Trp(Boc)-Arg(Pbf)-Thr(tBu)-Glu(OtBu)-Ala-Pro-Ser(tBu)-Ala-Thr(tBu)-Gly-Gln-Ala-Ser(tBu)-Ser(tBu)-Leu-Leu-Gly-Gly-Arg(Pbf)-Leu-Leu-Gly-Gln-Ser(tBu)-Ala-Ala-Ser(tBu)-Cys(Trt)-His(Trt)-His(Trt)-Ala-Tyr(tBu)-Ile-Val-Leu-Cys(tBu)-Ile-Glu(OtBu)-Asn-Ser(tBu)-Phe-Met-resin.

500 mg (0.1 ml) of Fmoc-Met-resin were suspended in 25 ml of DMF and after 2 hours loaded into the reaction column, then the operative cycle reported in example 1 was repeated, using the suitably activated Fmoc-amino acid for each cycle, in the order indicated in the sequence reported above.

960 mg of protected resin-hexatetracontapeptide were obtained.

### EXAMPLE 8

Tyr-Cys(Trt)-Glu-Thr-Trp-Arg-Thr-Glu-Ala-Pro-Ser-Ala-Thr-Gly-Gln-Ala-Ser-Ser-Leu-Leu-Gly-Gly-Arg-Leu-Leu-Gly-Gln-Ser-Ala-Ala-Ser-Cys(Trt)-His-His-Ala-Tyr-Ile-Val-Leu-Cys(tBu)-Ile-Glu-Asn-Ser-Phe-Met.

960 mg of protected resin-hexatetracontapeptide were treated with 200 ml of a mixture having the following composition: TFA 80%; 5% H₂O; 2.5% ethanedithiol; 2.5% phenol and 5% thioanisole. The mixture was reacted for 3 hours with occasional stirring, then the procedure of example 2 was followed.

The crude product was purified by semipreparative HPLC as described above, to obtain 98 mg of pure, non oxidized hexatetracontapeptide.

[α]²⁰_{D}−48.9° (c=0.1 water).

mass spectrum: molecular peak (M+1)=5455 Da.

amino acid analysis: Asp=0.96 (1); Thr=2.97 (3); Ser=5.87 (6); Glu=5.03 (5); Pro=0.93 (1); Gly=4.12 (4); Ala=5.88 (6); Cys=2.84 (3); Val=1.05 (1); Met=0.91 (1); Ile=2.11 (2); Leu=4.82 (5); Tyr=1.94 (2); Phe=1.20 (1); His=1.91 (2); Arg=2.03; Trp=0.95 (1).

### EXAMPLE 9

Tyr-Cys-Glu-Thr-Trp-Arg-Thr-Glu-Ala-Pro-Ser-Ala-Thr-Gly-Gln-Ala-Ser-Ser-Leu-Leu-Gly-Gly-Arg-Leu-Leu-Gly-Gln-Ser-Ala-Ala-Ser-Cys-His-His-Ala-Tyr-Ile-Val-Leu-Cys-(tBu)-Ile-Glu-Asn-Ser-Phe-Met.

98 g (17.96 mmoles) of non oxidized hexatetracontapeptide were dissolved in 200 ml of 75% methanol and then added drop by drop and under stirring with a solution of 10 mg of iodine in 30 ml of 75% methanol. After reacting the mixture for 3 hours at room temperature, a 10% ascorbic acid aqueous solution was added until complete decolourization of iodine. Methanol was thoroughly evaporated off under vacuum and the remaining aqueous solution was freeze-dried. The resulting crude peptide was finally purified by semipreparative HPLC, in the conditions described above.

11 mg of pure oxidized hexatetracontapeptide were obtained.

[α]²⁰_{D}−24° (c=0.05 water).

mass spectrum: molecular peak (M+1) 4968 Da.

Amino acid analysis: Asp=1.03 (1); Thr=2.87 (3); Ser=:5.91 (6); Glu=4.99 (5); Pro=0.95 (1); Gly=3.87 (4); Ala=5.91 (6); Cys=2.79 (3); Val=0.97 (1); Met 0.93 (1); Ile=2.21 (2); Leu=4.92 (5); Tyr=1.89 (2); Phe=1.09 (1); His=1.89 (2): Arg=1.99 (2); Trp=0.87 (1).

### EXAMPLE 10

D-Phe-Gln-Pro-Val-Leu-His(Trt)-Leu-Val-Ala-Leu-Asn-Ser(tBu)-Pro-Leu-Set (tBu)-Gly-Gly-Met-D-Arg(Pbf)-Gly-Ile-D-Arg(Pbf)-Gly-Ala-Asp(OtBu)-D-Phe-Gln-Cys(Acm)-D-Phe-Gln-Gln-Ala-D-Arg(Pbf)-Ala-Val-Gly-Leu-Ala-Gly-Thr(tBu)-Phe-D-Arg(Pbf)-Ala-Phe-resin.

Analogously to example 1, using Fmoc-D-Arg(Pbf) instead of Fmoc-Arg(Pbf), 1187 mg of protected resin-tetratetracontapeptide were obtained starting from 500 mg of Fmoc-Phe-resin suspended in 25 ml of DMF.

### EXAMPLE 11

D-Phe-Gln-Pro-Val-Leu-His-Leu-Val-Ala-Leu-Asn-Ser-Pro-Leu-Ser-Gly-Gly-Met-D-Arg-Gly-Ile-D-Arg-Gly-Ala-Asp-D-Phe-Gln-Cys(Acm)-D-Phe-Gln-Gln-Ala-D-Arg-Ala-Val-Gly-Leu-Ala-Gly-Thr-Phe-D-Arg-Ala-Phe.

Analogously to example 2, from 1187 mg of product of example 10.83 mg of pure peptide were obtained having the following characteristics:

mass spectrum: molecular peak (M+1): 4778 Da.

[α]²⁰_{D}: −55.8° (c=0.5 water).

### EXAMPLE 12

His(Trt)-Leu-Val-Ala-Leu-Asn-Ser(tBu)-Pro-Leu-Ser(tBu)-Gly-Gly-Met-Arg(Pbf)-Gly-Ile-Arg(Pbf-Gly-Ala-Asp(OtBu)-Phe-Gln-Cys(Acm)-Phe-Gln-Gln-Ala-Arg(Pbf)-Ala-Val-Gly-Leu-Ala-Gly-Thr(tBu)-Phe-Arg(Pbf)-Ala-Phe-Leu-Ser(tBu)-Ser(tBu)-Arg(Pbf)-Leu-Gln-Asp(OtBu)-Leu-Tyr(tBu)-Ser(tBu)-Ile-Val-Arg(Pbf)-Arg(Pbf)-Ala-resin.

Analogously to example 1, starting from the suitable Fmoc-amino acids, 1285 mg of protected resin-tetrapentacontapeptide were obtained from 500 mg of Fmoc-Ala-resin in 25 ml of DMF.

### EXAMPLE 13

His-Leu-Val-Ala-Leu-Asn-Ser-Pro-Leu-Ser-Gly-Gly-Met-Arg-Gly-Ile-Arg-Gly-Ala-Asp-Phe-Gln-Cys-Phe-Gln-Gln-Ala-Arg-Ala-Val-Gly-Leu-Ala-Gly-Thr-Phe-Arg-Ala-Phe-Leu-Ser-Ser-Arg-Leu-Gln-Asp-Leu-Tyr-Ser-Ile-Val-Arg-Arg-Ala.

Analogously to example 2, from 1285 mg of the compound of example 12, 125 mg of pure peptide were obtained, having the following characteristics:

mass spectrum: molecular peak (M+1): 5953 Da.

[α]²⁰_{D}: −62.6° (c=0.45 water).

### EXAMPLE 14

Endothelial human cells EA.hy.926 were grown in DMEM supplemented with 10% fetal bovine serum (FBS) and with the appropriate concentrations of glutamine and antibiotics. Before the experiments, the cells had been deprived of serum for 24 hours in 0.1% FBS.

EA.hy.926 cells migration has been evaluated by chemotaxis test in a 48 well Boyden chamber using polycarbonate filters of 12 μm porosity pre-treated with a 10 μg/ml type I collagen solution. The cells were added to the wells of the superior chamber at the density of 15.000 cells/well in the presence or in the absence of the endostatin fragment 6-49 or of human endostatin. The chemotactic stimulus, represented by a conditioned medium obtained by a culture of glioma cells, was added to the lower chamber. After 4 hours of incubation at 37° C., non migrated cells were removed by a scraper and the filter was colored with Diff Quick. Migrated cells were then counted at a 400×magnification in 6 different fields.

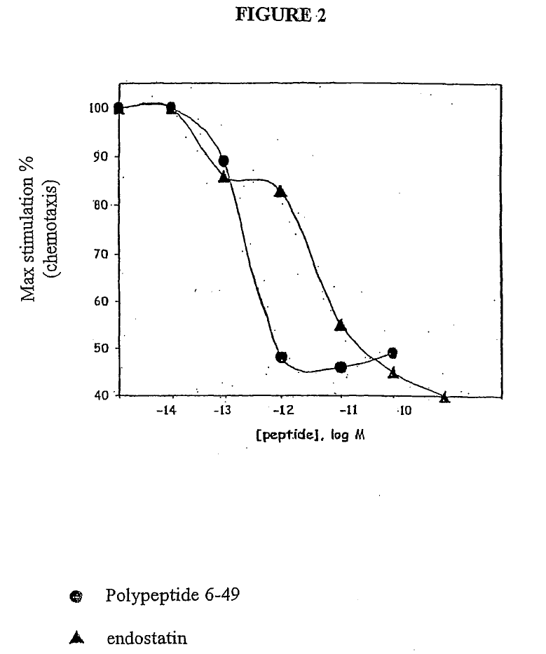

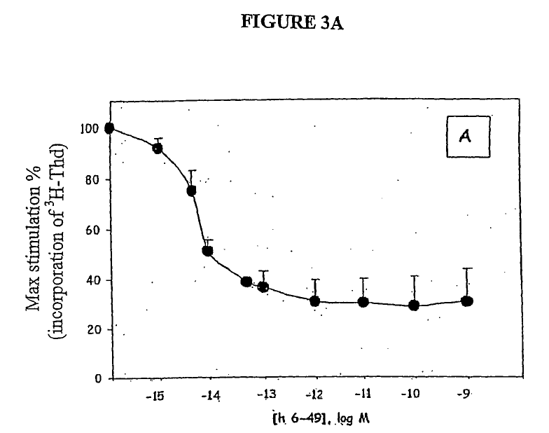

The results, reported in FIG. 2, are expressed as a percentage of the maximal migration induced by the conditioned medium in the presence of the peptide or of endostatin.

Peptide 6-49 causes maximal inhibition of cell migration of about 60% starting from the concentration of 10⁻⁹ M, with an ID₅₀ of 3×10⁻¹³, while endostatin determines a maximal inhibition of 70% at 10⁻⁹ M, with an ID₅₀ of5×10⁻¹² M.

### EXAMPLE 15

EA.hy.926 cells were inoculated in 96 well-plates and, after being deprived of serum for 24 hours, were 'stimulated with 10% FBS in the presence or in the absence of different concentrations of the peptide 6-49 or of endostatin for further 24 hours. Tritiated tymidine (1 μCi/well) was added during the last 6 hours of incubation. The cells were then extracted in 10% TCA and the radioactivity incorporated in the TCA-insoluble fraction was determined after solubilization in 0.5 M NaOH.

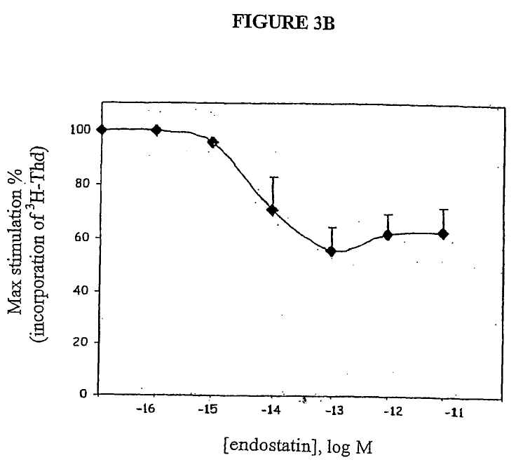

The results, reported in FIGS. 3a and 3b, are expressed as the percentage of the maximal stimulation induced by 10% serum in the absence of the drug.

Peptide 6-49 induces maximal inhibition of DNA synthesis of about 80% starting from the concentration of 10⁻¹² M, with an ID₅₀ of 5×10⁻¹⁵ M. Human endostatin induces maximal inhibition of DNA synthesis of about 55% starting from the concentration of 10⁻¹³ M, with an ID₅₀ of 10⁻¹⁴.

### EXAMPLE 16

The formation of tubular structures, similar to capillaries, was evaluated by seeding the endothelial cells on a Matrigel carrier, a re-constructed basal membrane, with the characteristic of being liquid at 4° C. and of undergoing polymerization at 37° C. forming a tri-dimensional gel. Proangiogenic factors, such as Fibroblast Growth Factor (FGF) or Vascular Endothelial Growth Factor (VEGF), were added to the medium in the presence of the peptide 9-49 and the plate's were incubated at 37° C. under 5% CO₂ atmosphere. Tubules formation was monitored observing the cells with an inverted microscope and, after recording the image by means of photography, a quantification was performed by evaluating the area occupied by the cells and by the capillaries network.

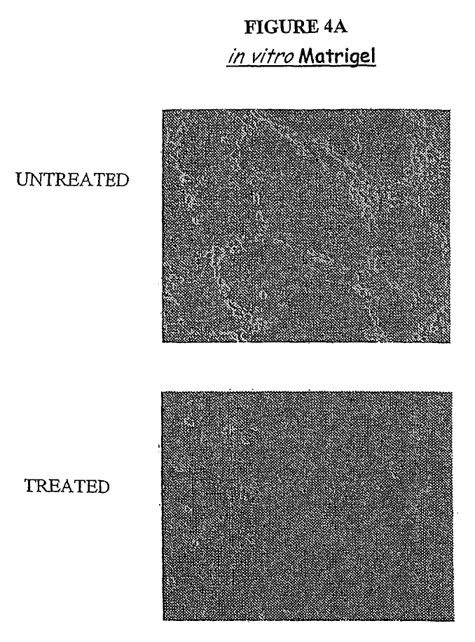

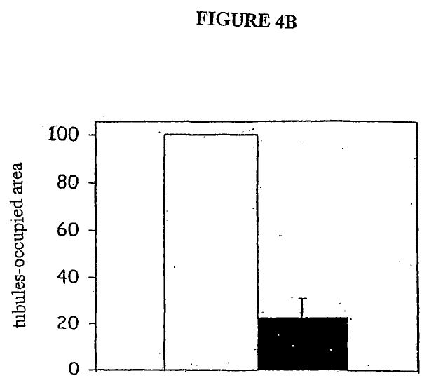

Upon observation under the microscope, it is evident that the peptide of invention is able to inhibit the capacity of endothelial cells to form tubular structures similar to capillaries, which on the contrary are clearly evident in the untreated control cells (FIG. 4A). In FIG. 4B, a quantitative analysis of the effect, carried out by a proper software (NIH Image), is reported.

Treatment with the fragment 6-49 reduces tubules formation to 23% compared with controls, considered as 100%.

### EXAMPLE 17

500 ml of Matrigel containing FGF 2 ng/ml and heparin 36 U/ml were inoculated s.c. in the abdominal region of male mice C57/b16, of age from 6 to 10 weeks. Where indicated, fragment 6-49 was added to the solution of Matrigel at concentrations of 1 and 10 μg/mouse. Six animals were used in each experiment. After 4 days, the gelatinized Matrigel pellet was recovered and the amount of hemoglobin therein was measured by means of a commercial kit based on Drabkin's method (Signia Aldrich).

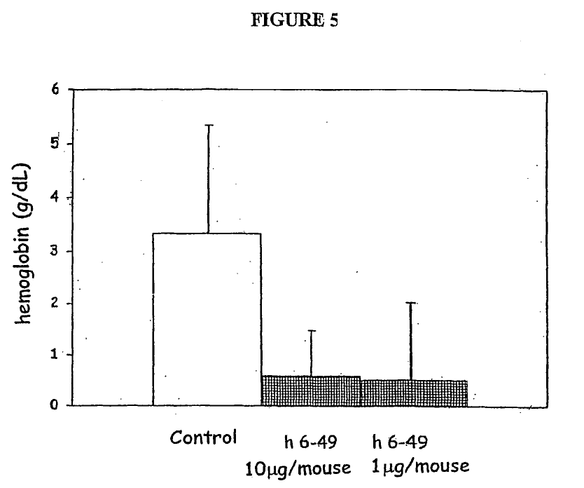

As it can be observed in FIG. 5, which shows the data obtained in three independent experiments, the fragment 6-49, already at the dose of 1 mg/mouse, is capable of reducing hemoglobin levels in the Matrigel pellets, which indicates a decreased formation of vessels in animals treated with the fragment compared with controls.

1. Peptides comprising 20 to 50 amino acids with sequences corresponding to the 6-179 sequence of endostatin, the salts and the non toxic derivatives thereof.

2. Peptides as claimed in claim 1 with sequence ranging from the amino acids 6 to 92 of the human sequence.

3. Peptides as claimed in claim 2 with sequence ranging from the amino acids 6 to 64.

4. Peptides as claimed in any one of the above claims selected from those with sequence 6-49, 11-64, 50-92, 93-133 or 134-179 of the sequence of human endostatin.

5. Peptide as claimed in claim 4 having the sequence 6-49 of human endostatin.

6. Pharmaceutical compositions containing the peptides of claims 1-5 in admixture with a suitable carrier.

7. Use of the peptides of claims 1-5 for the preparation of medicaments with antiangiogenic activity.


SEQUENCE LISTING (5 sequences)

SEQ ID NO: 1 - protein, 44 aa, Homo sapiens
FQPVLHLVALNSPLSGGMRGIRGANFQCFQQARAVGLAGTFRAF

SEQ ID NO: 2 - protein, 43 aa, Homo sapiens
LSSRLQDLYSIVRRADRAAVPIVNLKDELLFPSWEALFSGSEG

SEQ ID NO: 3 - protein, 41 aa, Homo sapiens
PLKPGARIFSFDGKDVLRHPTWPQKSVWHGSDPNGRRLTES

SEQ ID NO: 4 - protein, 46 aa, Homo sapiens
YCETWRTEAPSATGQASSLLGGRLLGQSAASCHHAYIVLCIENSFM

SEQ ID NO: 5 - protein, 46 aa, Homo sapiens; identical to SEQ ID NO: 4
  DISULFID (2)..(32)